(19) United States
(12) Patent Application Publication
(10) Pub. No.: US 2024/0142429 A1
(43) Pub. Date: May 2, 2024

(54) CHEMICAL MONITORING UNIT AND METHODS

(71) Applicant: General Galactic Systems, Inc., Monte Sereno, CA (US)

(72) Inventors: Justin Miller, San Diego, CA (US); Josh Kennedy, La Jolla, CA (US); Scott Eklund, San Diego, CA (US)

(21) Appl. No.: 18/499,346

(22) Filed: Nov. 1, 2023

Related U.S. Application Data
(60) Provisional application No. 63/421,443, filed on Nov. 1, 2022.

(51) Int. Cl. G01N 33/18

(52) U.S. Cl. CPC G01N 33/18

(57) ABSTRACT

Disclosed is a pool water monitoring system and monitoring techniques. A pool monitoring system may include a monitoring unit and a computer application. The monitoring unit includes a surface based unit, a submersible cable connection assembly, and a submerged sensing device. The submerged sensing device is configured to measure water parameters, a submersible cable connection assembly connects the surface based unit and the submerged sensing device together. The surface based unit is configured to receive the measured water parameters via the submersible cable connection. The surface base unit is operable to wirelessly communicate with the computer application of the client device. The computer application is operable to receive the measured water parameters transmitted by the surface base unit; evaluate the measured water parameters; and present indications, on a touchscreen of a client device, of the measured water parameters and evaluation results of the measured water parameters.

### RELATED APPLICATION

The application claims the benefit of U.S. Provisional Patent Application No. 63/421,433, filed on Nov. 1, 2022, the contents of which are incorporated herein by reference in their entirety.

### FIELD

The disclosed subject matter relates to devices, systems, and methods for monitoring and adjusting chemical levels in a liquid and, more particularly, to a device, a system, and methods for monitoring and adjusting chemical levels in, for example, a swimming pool, a hot tub, and the like.

### BACKGROUND

Automated water care is configured and operable to maintain water properties (e.g., pH, water temperature, oxidation-reduction potential (ORP), and so forth) within acceptable levels of quality in, for example, swimming pools, diving pools, hot tubs, and other sports, recreational, and therapeutic bodies of water. Advantageously, automated water care should maintain water clarity and prevent waterborne illnesses.

Some pool owners may engage commercial professionals to maintain the water quality of their pools; other owners may manage water quality and chemical levels themselves, using off-the-shelf testing kits and adding a host of liquid and solid chemicals, as necessary. Although each of these techniques has its benefits (e.g., cost, simplicity, and the like), they are also flawed.

Attempts to automate water care have met with some degree of success. However, early systems were both complex and costly. Prior water-borne sensing systems helped to reduce the cost and simplify the water care process; however, the system had problems with accuracy and reliability. Prior systems, such as the one described in U.S. Pat. No. 9,834,485, included a water-borne sensor that was structured and arranged to measure ORP, pH levels, and water temperature. These prior systems also relied on calibration test strips to measure, for example, pH, chlorine, cyanuric acid, hardness, and total alkalinity. The test strip would then be placed on a marked area of a calibration card and an image of the test strip and calibration card would be taken and transmitted to a remote technician and/or a remote server configured and operable to process the transmitted images and to return values for the tested chemical (e.g., values related to pH, chlorine, cyanuric acid, water hardness, and total alkalinity).

The prior systems relied on a connection between the water-borne sensor device and a user's mobile device, such as the pool owner's mobile device, and/or a hub device that was connected to the internet. Obstructions in a poolside environment, such as a side of the pool, a water pathway to a filtering system, ladders, and other sources of interference can cause connectivity and data integrity issues which may lead to inefficient and erroneous implementation of a water quality treatment plan.

### SUMMARY

In a first aspect, an embodiment of the disclosed subject matter relates to a monitoring unit for monitoring the condition of pool water. The monitoring device includes a sensing device, a surface base unit, and submersible cable connection assembly. The sensing device is configured to be submerged in the pool water and may include one or more water parameter sensors. The one or more water parameter sensors are operable to measure at least one parameter of the water. The surface base unit is disposed to be out of the pool water, and includes electronic components configured to receive a measurement data of at least one parameter of the pool water from the sensing device and to wirelessly transmit the measurement. The submersible cable connection assembly may be removably coupled to the surface base unit outside of the pool water and permanently coupled to the sensing device that is submerged in the pool water.

In another aspect, another embodiment of the pool water monitoring system includes a monitoring unit and a computer application. The monitoring unit includes a surface based unit, a submersible cable connection assembly, and a submerged sensing device. The submerged sensing device is configured to measure water parameters, the surface based unit is coupled to a first end of the submersible cable connection assembly, and the submerged sensing device is coupled to a water-tight, second end of the submersible cable connection assembly. The surface based unit is configured to receive the measured water parameters via the submersible cable connection. The computer application is executable on a client device. The surface base unit is operable to wirelessly communicate with the computer application. The client device includes a touchscreen communicatively coupled to the computer application. When the computer application is executing on the client device, the client device is operable to receive the measured water parameters transmitted by the surface base unit; evaluate the measured water parameters; and present indications on the touchscreen of the measured water parameters and evaluation results of the measured water parameters.

### BRIEF DESCRIPTION OF THE FIGURES

In the drawings, like reference characters generally refer to the same parts throughout the different views. Also, the drawings are not necessarily to scale, emphasis instead generally being placed upon illustrating the principles of the disclosed subject matter.

FIG. 1 illustrates an exemplary water monitoring unit, in accordance with an embodiment of the disclosed subject matter.

FIG. 2 illustrates an exploded view of an exemplary surface base unit usable in the water monitoring unit of FIG. 1, in accordance with an embodiment of the disclosed subject matter.

FIG. 3 illustrates a block diagram of an exemplary water monitoring system, in accordance with an embodiment of the disclosed subject matter.

FIG. 4A shows an exemplary pole-mounted version of the water monitoring unit of FIG. 1, in accordance with an embodiment of the disclosed subject matter.

FIG. 4B shows an exemplary deck-mounted version of the water monitoring unit of FIG. 1, in accordance with an embodiment of the disclosed subject matter.

FIG. 5A illustrates an exemplary graphical user interface presenting monitored evaluation results in accordance with an embodiment of the disclosed subject matter.

FIG. 5B illustrates an exemplary graphical user interface presenting expanded monitored evaluation results and action item messages, in accordance with an embodiment of the disclosed subject matter.

FIG. 5C illustrates an exemplary graphical user interface presenting information and instructions for correcting the action item, in accordance with an embodiment of the disclosed subject matter.

FIG. 6 shows an illustrative embodiment of a water monitoring system, in accordance with an embodiment of the disclosed subject matter.

FIG. 7 illustrates a flowchart of an exemplary algorithm executed by an embodiment of a water monitoring system as described herein.

### DETAILED DESCRIPTION

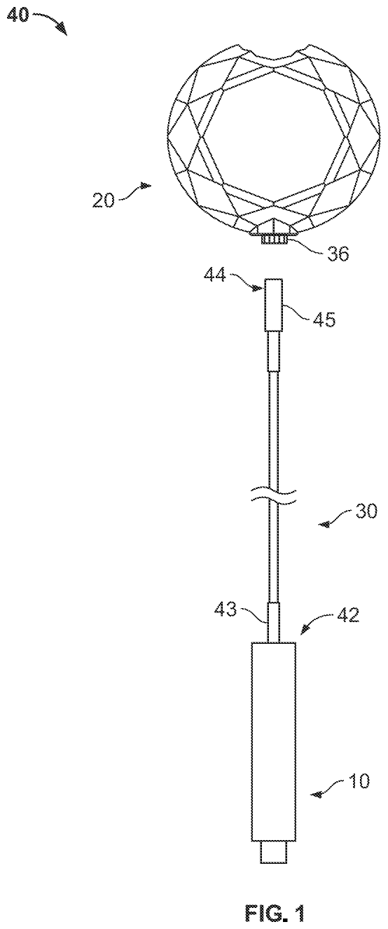

Referring to FIGS. 1-3, a water monitoring system 100 for monitoring water parameters, such as temperature, and chemical parameters in a body containing the water, such as a swimming pool, a spa, a hot tub, and/or other (e.g., synthetic) structures for holding and retaining water is presented. The various bodies that contain the water may also be generally referred to as a “swimming pool” or “pool,” and the terms may be used interchangeably with reference to a spa, a hot tub, and the like. More specifically, the body containing the water is intended to be for use by people to sit, swim, and partake in other water-related activities. Additionally, the “water” is generally refers to as pool water herein, which is intended to include water in spas, hot tubs, and the like. Advantageously, the water monitoring system 100 is structured and arranged to notify a user and/or interested third parties (e.g., maintenance personnel) about the current state of the pool water in the swimming pool and to identify any remedial actions that may need the user's or third party's attention. Although the disclosed subject matter is described and shown in the various figures in connection with a particular use with a swimming pool, this is done for the purpose of conciseness and brevity rather than to limit the disclosed subject matter. Additionally, the disclosed water monitoring system 100 via its multi-part configuration (i.e., surface base unit and submerged sensing device) is operable to maintain consistent communication connectivity with user devices and network components thereby allowing near continuous or on demand access to measurement data and remedial action recommendations related to a water-quality treatment plan for the swimming pool.

FIG. 1 shows an exemplary embodiment of a monitoring unit 40 that is configured and operable to be placed in and near a swimming pool for the purpose of monitoring a myriad of water parameters, such as, for example, water temperature, water pH, total hardness, total alkalinity, chlorine level, bromine level, oxygen reduction potential (ORP), cyanuric acid level, total dissolved solids, phosphate levels, metal levels, and so forth.

In some implementations, as shown in FIG. 1, the monitoring unit 40 may include a submersible sensing device 10, a surface base unit 20, and a connection cable assembly 30. The submersible sensing device 10 and the surface base unit 20 are electrically-, electronically-, and operatively coupled to one another by the connection cable assembly 30. The submersible sensing device 10 may include one or more water parameter sensors operable to measure at least one parameter of the water. The types and configurations of the water parameter sensors are known and may be configured to be disclosed in the submersible sensing device 10. In some implementations, the submersible sensing device 10 is configured to be placed within the pool water (e.g., submerged) and is operable to monitor and to provide data on, for the purpose of illustration rather than limitation, one or more of: water temperature, water pH, total water hardness, total water alkalinity, chlorine level, bromine level, oxygen reduction potential (ORP), cyanuric acid level, total dissolved solids, phosphate levels, or metal levels. It is also envisioned the submersible sensing device 10 may be operable to deliver less information, such as, specifically, water temperature, water pH and ORP.

Of course, the submersible sensing device 10 may be configured with a number of different sensors relevant to the quality and status of the water being monitored and be operable to provide more or less information than described above. In addition, the sensing device 10 may be configured and operable to measure chemical parameters (e.g., pH, alkalinity, and the like) and/or parameters environmental (e.g., water temperature, air temperature, and the like) at a desired reading frequency (e.g., every second, every minute, every 30 minutes, every hour, twice a day, once per day, and so forth). Reporting frequency of the measured (or sensed) data, such as the measured chemical and/or environmental parameters, may also be selectively adjustable depending on time of the day. For example, the sensing device 10 may take more samples and communicate the resulting data during periods of greater pool use (e.g., during the morning and afternoon) and fewer samples during periods of limited pool use (e.g., after midnight). Advantageously, the sampling frequency and reporting frequency may be separately controlled, such that, while sampling may be done more frequently (e.g., every 10 minutes), reporting of the accumulated measured data may be performed less frequently (e.g., every two hours). Reporting frequency may also be adjusted to take into account the time of day (or night), when reporting is not desirable or practicable (e.g., during pool owner working hours). “Measured data” may generally refer to raw data obtained by the respective sensors in sensing device 10, processed data that was raw data that has been interpreted via circuitry to provide a measurement of a chemical or environmental parameter being sensed, or a combination of both.

In addition to having scheduled sampling, testing, and reporting, the monitoring unit 40 may, in some implementations, enable a user to perform an “on demand” sampling. For example, the monitoring unit 40 may include an input device (e.g., an ON button not shown but located in surface base unit 20) that, when pressed by the user, causes the sensing device 10 to take and process a sample of water, and transmit measured data of the “on demand” test to a client device (shown in another example).

The surface base unit 20 is configured to be disposed out of the pool water. The surface base unit 20 may include electronic components (shown in other examples) configured to receive measurement data of at least one parameter of the pool water from the sensing device 10 and to wirelessly transmit the measurement data.

Although much of the data collected and transmitted by the surface base unit 20 and detected by sensing device 10 has to do with properties associated with the state of the water, the monitoring unit 40 may also be configured and operable to accumulate and process data associated with the monitoring unit 40 itself, for example, the battery level, the signal strength (e.g., with respect to the monitoring unit's connectivity to a client device and/or a communication network), and so forth.

The connection cable assembly 30 may be configured and operable to couple the sensing device 10 electrically and electronically to the surface base unit 20. The cable connection assembly 30 is submersible and removably coupled to the surface base unit 20 that is located outside of the pool water at a first end and coupled via a waterproof coupling to the sensing device 10 that is submerged in the pool water at a second end. In an embodiment, the connection cable assembly 30 comprises a pair of (e.g., 8-pin) IP67 or IP68 cable connectors 43, 45 that have been fixedly attached (e.g., soldered) to the connection cable 30 at a distal end 42 (also referred to as a first end) and a proximal end 44 (also referred to as a second end) of a length (e.g., four-foot, six-foot, eight-foot, and the like) of a submersible cable. Advantageously, the waterproof coupling of the connection cable assembly 30 may be formed by overmolding a water-tight material over the cable connector 43 where the connection cable assembly 30 joins the cable connector 43 at the distal end 42. Similarly, the cable connector 45 may be overmolded with the connection cable assembly 30 at the proximal end 44. Overmolding ensures that the cable connectors 43, 45 remain waterproof, and, by extension, so do the connection cable assembly 30 and the sensing device 10.

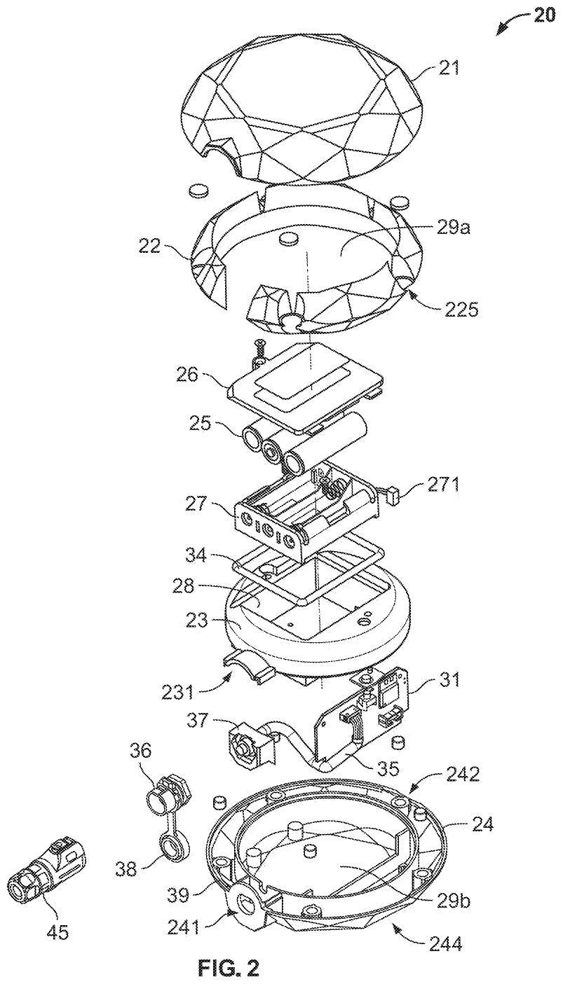

Referring to FIG. 2, an illustrative embodiment of the surface base unit 20 is shown. In some implementations, the surface base unit 20, which may also be referred to herein as a monitoring unit, may include an upper housing portion 21, an inner annular portion 22, an inner top portion 23, and a lower housing portion 24 that are selectively and releasably attachable to one another using, for example, fastening devices (e.g., screws, bolts, snaps, tabs, springs, and the like). To ensure that the mechanical components and electrical circuitry of the surface base unit 20 are protected from water and the external environment, the upper housing portion 21, inner annular portion 22, inner top portion 23, and lower housing portion 24 may be structured and arranged to provide a water-tight, or substantially water-tight, seal when operatively coupled together. In some embodiments, a sealing device 39, such as, an O-ring, a gasket, or the like, may be included in a groove or the like in the upper housing portion 21, the lower housing portion 24, or both.

In some examples, the upper housing portion 21 is configured to removably couple to the inner annular portion 22 of the surface base unit 20 to conceal fixation openings 225 formed in the inner annular portion 22.

Advantageously, when coupled to one another, the upper housing portion 21 and the inner annular portion 22 are structured and arranged to provide a first plenum space 29a while, when operatively coupled to one another, the lower housing portion 24 and the inner top portion 23 are structured and arranged to provide a second plenum space 29b. The first plenum space 29a and second plenum space 29b are configured to accommodate the electrical circuitry and mechanical components of the base unit 20.

For example, in some implementations, the inner top portion 23 and inner annular portion 22 form a tight interference fit with, respectively, the lower housing portion 24 and the upper housing portion 21. The resulting first plenum space 29a is dimensioned and configured to provide space for accommodating, inter alia, portions of the inner top portion 23, while the resulting second plenum space 29b is dimensioned and configured to provide space for accommodating, inter alia, a printed circuit board (PCB) 31 (also referred to as electrical circuitry), an inner data port connection 37, and a data bus 35 that electronically couples the PCB 31 and the inner data portion connection 37. In some implementations, the data port water barrier 37 is coupled to a data port connector 36 (e.g., IP68 eight-pin panel mount) for coupling the base unit 20 to a cable connector 45 of a connection cable assembly 30. The data port connector 36 couples electrically to the PCB 31 by connecting to the data bus 35. The cover 38 may be plastic, rubber, or the like, and configured to provide a watertight seal when the cable connector 45 of the submersible cable connection assembly (shown in another example) is removed from the surface base unit 20. For example, when the data port connector 36 is not coupled to the cable connector 45 of the connection cable assembly 30 (due to maintenance or draining of the pool), the cover 38 may be placed over the data port connector 36 to provide a watertight seal.

The annular portion 22 may be configured as a circular arc having an opening operable to allow space for the data port water barrier 37 to be accessible to an outer data port connector 36. The inner annular portion 22 may also be further configured with one or more fixation openings 225 to enable fastening devices (e.g., screws, snap rivets, and the like) to be inserted through the fixation openings 225 and into securing points 242 in the lower housing portion 24.

Advantageously, a power source housing area 28 may be formed in the inner top portion 23. In some embodiments, the power source housing area 28 is configured and dimensioned to accommodate a power source 25, a power connection bus 27, and an insulation device 34. The power source 25 may be shown as a plurality of batteries, but the power source 25 may be a battery pack, a battery, a solar cell, a combination of power sources, or the like). As shown in FIG. 2, a cover 26 may be used to releasably retain the power source 25, the power connection bus 27, and the insulation device 34 within the power source housing area 28. The power connection bus 27 is configured to receive the power source 25, such as one or more batteries, and includes a power connector 271 operable to connect to other components of the surface base unit 20, such as the surface base unit circuitry (i.e., PCB) 31 to supply power from the power source 25. In some implementations, the surface base unit circuitry 31 may contain electronics or electronic that may receive signals from water parameter sensors within the sensing device 10. For example, the surface base unit electronic components may include one or more of a processing device (e.g., a processor, microprocessor, logic circuitry); memory (e.g., RAM, ROM, and the like) for temporary storage and/or permanent storage of measured data, as well as permanent storage for a plurality of driver programs, algorithms, programming instructions, applications, and the like, and one or more wireless communication transceivers (e.g., Bluetooth®, Wi-Fi, cellular (e.g., LTE or 5G)). In a further example, the processing device of the surface base unit circuitry 31 may be electrically coupled to one or more sensors within the sensing device 10 and be operable to store measured data in the memory.

In a further example, the inner top portion 23 may include an inner flange 231 operable to fit into the opening in the annular portion 22. The lower housing portion 24 may include a lower flange 241 that is corresponds to inner flange 231 to form a structure to secure the inner data port connection 37.

The surface base unit 20 may also include an attachment area 244 as part of the lower housing portion 24 configured to receive attachment devices, such as tie wraps, hose clamps, or the like, enabling the surface base unit 20 to be affixed to ladders, diving board supports, sides or walls of a hot tub or spa, and/or other structures of a body holding the water.

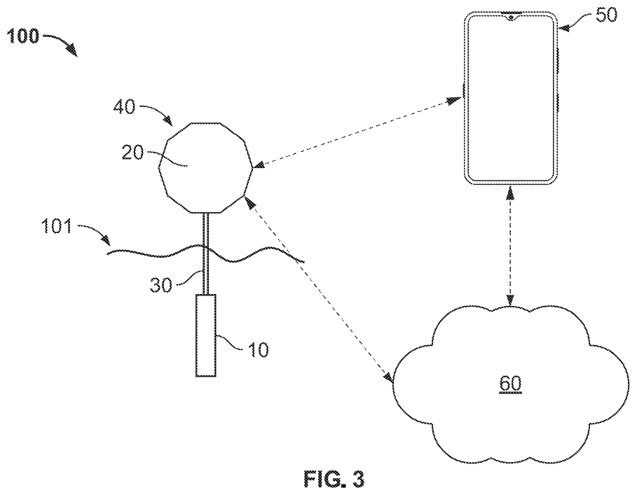

FIG. 3 illustrates a block diagram of an exemplary water monitoring system, in accordance with an embodiment of the disclosed subject matter. The exemplary water monitoring system 100 may include the monitoring unit 40 and a client device 50. The water monitoring system 100 may also be configured to communicate with a communication network 60. The sensing device 10 and a portion of the submersible cable connection assembly 30 may be submerged in the pool water 101. The communication network 60 may be a connected home/house network, a cellular network, data network, a combination of communication/data networks, or the like. The surface base unit 20 and the client device 50 may be operable to connect to the communication network 60 via one or more communication protocols as described herein. More specifically, as shown in FIG. 3, one or more of the electronics or electronic components of the PCB 31 may be a transceiver configured and operable to connect wirelessly (e.g., using Wi-Fi) to the user's (or, alternatively, a third party's) client device 50 via the communication network 60. Those of ordinary skill in the art can appreciate that a variety of protocols (e.g., Bluetooth, LTE, or 5G, Wi-Fi, X-10, ad hoc network protocols, or the like) for the wireless connection between the monitoring unit 40 and the client device 50 are possible.

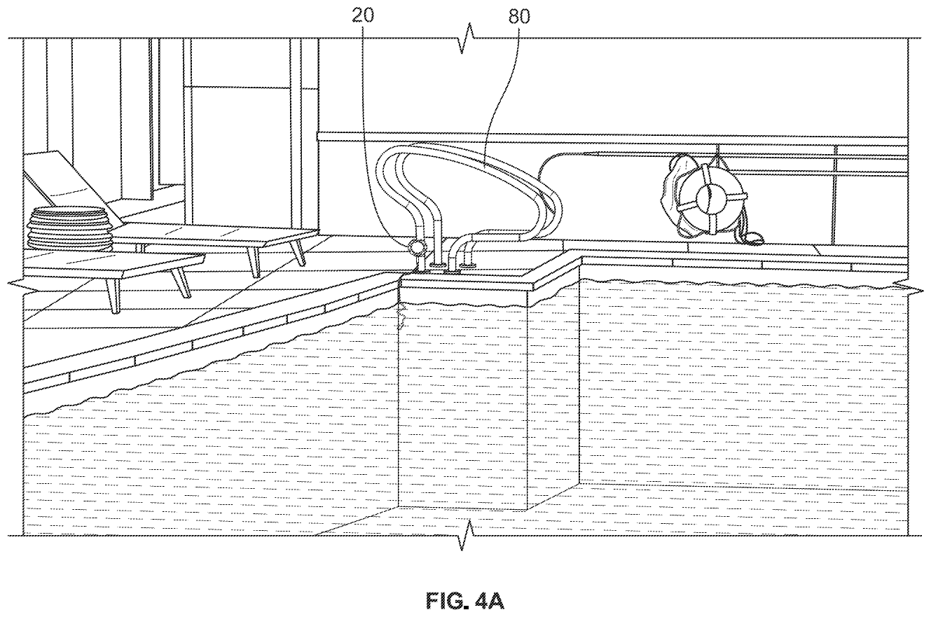

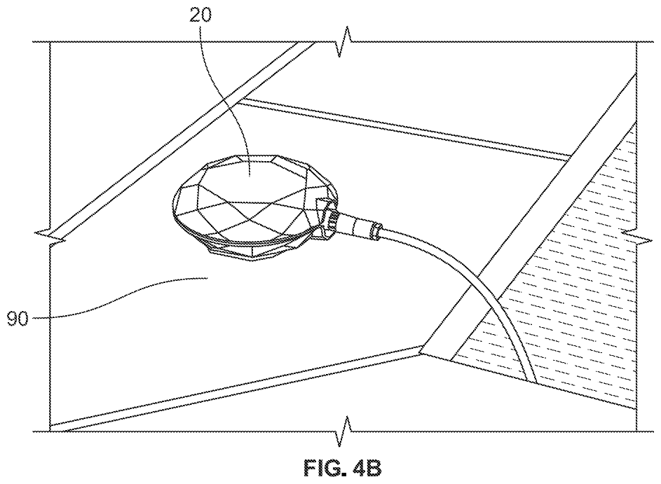

In some implementations, as shown in FIG. 4A, the surface base unit 20 may be mounted to a pole 80 adjacent or near the edge of the pool, hot tub, spa, or body of water being monitored. As shown, the pole 80 may be part of a ladder that enables users to enter and exit the pool. The surface base unit 20 may be positioned in a vertical or substantially vertical orientation so the connection cable assembly 30 extends directly into the water being monitored. For example, the surface base unit 20 may include an attachment area (shown in an earlier example) configured to receive attachment devices enabling the surface base unit 20 to be affixed to the pole 80 as well as other structures of a body holding the pool water. For vertically mounting the base unit 20, a clip or bracket may be releasably or removably attached to the reverse or back side of the lower housing portion 24. Alternatively, as shown in FIG. 4B, the surface base unit 20 may be placed on a planar surface 90, in a vertical, substantially vertical, horizontal, or substantially horizontal orientation (e.g., the pool deck 90).

In some embodiments, the sensing device 10 may be installed “in line” within the plumbing (e.g., at the equipment pad) of the pool, spa, hot tub, and the like. For an “in line” implementation, while the sensing device 10 is still submerged, the system 100 may include an accelerometer or other flow measuring device (e.g., a flow meter, a pressure sensor, or the like) capable of determining that water is flowing through the conduit. The inline design may include valves to shut the water on/off. It may also include a sensor cap storage location that allows the sensor cap to be stored, for example, a threaded location where a user can screw the sensor cap, when it's not on the sensor. The sensor cap is used only when the sensor is being stored, not when it's in use. Those of ordinary skill in the art can appreciate that, if the water is not flowing through the conduit, then the water may be stagnant and, correspondingly, not representative of the state of the water. Whereas, if water is flowing through the conduit (as determined by the accelerometer or other measuring device), then the sampled water may be better representative of the state of the water.

Advantageously, the client device 50 may be operable to execute an application that is operable to communicate via one or more transceivers of the client device 50 with the surface base unit 20. Additionally, an output device (e.g., touchscreen or display device) on the user's (or, optionally, a third party's) client device 50 may selectively display parameters of the swimming pool transmitted from the monitoring unit 40 and, furthermore, may display notifications or alerts requiring supply level or user actions when water readings in the pool necessitate remedial action. For example, a notification may be pushed to the user's (or, optionally, a third party's) client device 50 instructing the user to add a particular chemical for maintaining a desired sanitary or chemical level in the swimming pool.

Furthermore, in order to provide a more complete picture of water chemistry to the pool owner, as well as the actions required to maintain water chemistry properly, the water monitoring system 100 enables users to leverage “water (e.g., chemistry) report” data provided from, for example, test strips, test kits, other sampling/testing devices, including data from water testing performed at local stores. Advantageously, the system may be configured to import such data to provide, in combination with the sensor data, a more complete picture of the water chemistry. Since much of these “water report” data may only be valid for a fixed period or length of time, the system may be operable delete “stale” data continuously depending on the reliability of the data over time. For example, some data may be used for a longer period or length of time (for example, to generate a history of measurements and remedial actions), while other data may only contribute to supplementing a more complete picture of the water chemistry for a short period of time. When measurement data becomes “stale,” in some implementations, the system 100 may alert the user that it would be desirable to obtain a new water report.

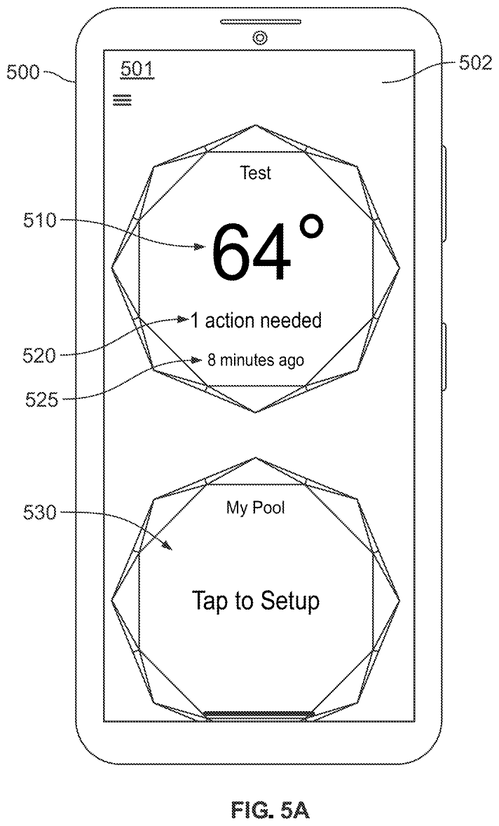

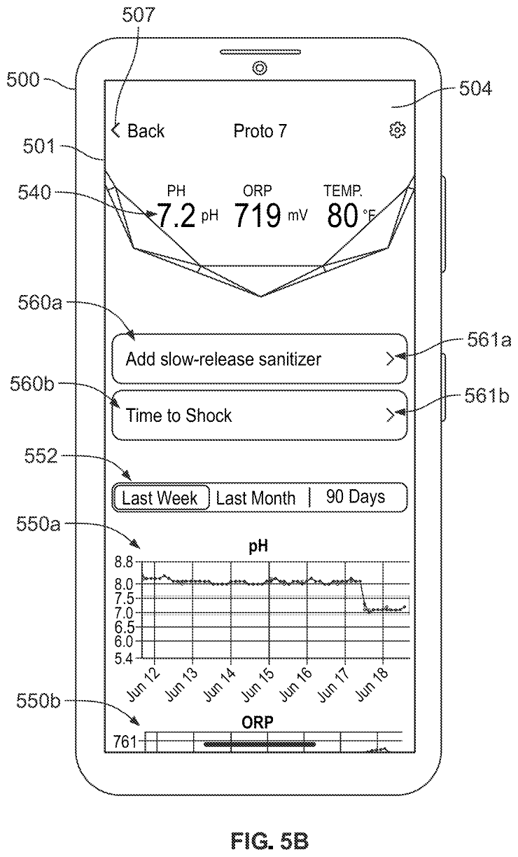

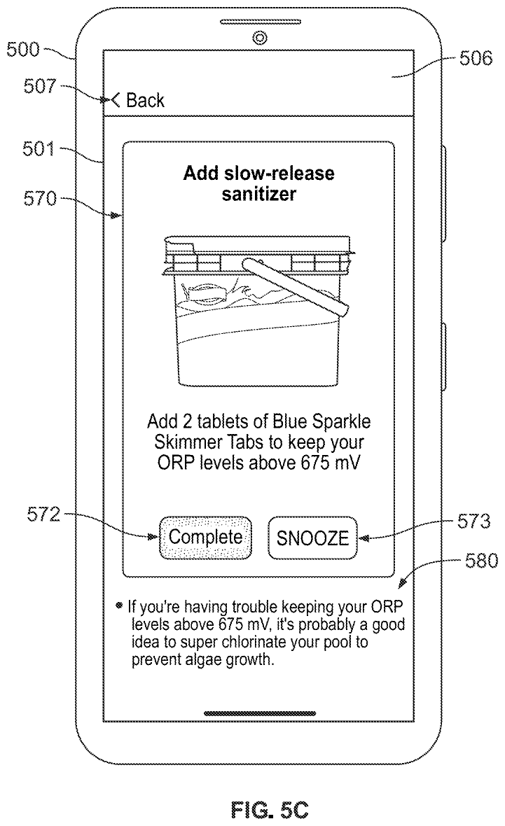

Exemplary graphical user interfaces that may be presented on the user's client device 500 are shown in FIGS. 5A through 5C. FIG. 5A shows an exemplary graphical user interface 502 presenting monitoring results and for setting up or adjusting settings of the water monitoring system 100. The touchscreen 501 of the client device 500 may include a first portion 510 of the graphical user interface 502. The first portion 510 may include a single parameter such as the water temperature, but may also include any other desired water parameters based on user preferences. A second portion 520 of the graphical user interface may provide an alert that some remedial action may be needed. Adjacent to the second portion 520 alert may be a timing reference 525. The timing reference 525 may indicate when the second portion 520 alert was presented, in the example, the timing reference 525 is shown as “8 minutes ago,” but the timing reference may be represented by a timestamp, a day indication, or the like. A third portion 530 of the graphical user interface 502 may include instructions for setting up the water monitoring system 100.

FIG. 5B shows an exemplary graphical user interface presenting a number of water parameters and, further, alerting the user of the need for remedial action. More specifically, in a first portion 540 of the graphical user interface 504, one or more water parameters (e.g., pH, ORP, temperature, alkalinity, clarity, or the like) may be displayed. In a second portion of the graphical user interface, historical readings of selected water parameters may be summarized in a graph, chart, or other data structure. For example, the water pH over time graph 550a, the ORP over time graph 550b, and water temperature over time may be displayed. The period of time over which the selected water parameters are presented may be user selectable. For example, selectable element 552 of the graphical user interface 504 may selected by a touch to the graphical user interface 504 and changed from a period of the last week, the last month, the last 90 days, or the like. In a third portion of the graphical user interface 504 above the second portion of the graphical user interface 504, remedial actions 560a and 560b necessitated by one or more of the water parameter readings, may be presented. The presented remedial actions 560a and 560b may include additional selectable elements, such as the right arrows 561a and 561b at the right of the text of the respective remedial actions 560a and 560b, that allow a user to obtain for more information regarding the respective remedial action.

FIG. 5C shows an exemplary screen shot providing the details for remediating the issue reported in the alert message. For example, in response to selection of the selectable element 561, the graphical user interface 504 may be operable to transition to a next window. In a first portion 570 of the graphical user interface 506 instructions for remediation may be shown. For example, an image of a remediation agent product, such as a brand of slow-release sanitizer may be presented as well as an amount (e.g., two tablets) and the brand name of the remediation agent (e.g., HTH® Blue Sparkle Skimmer™, or the like). The graphical user interface 506 may include remediation status buttons such as button 572 that indicates the instructions for remediation were followed or button 573 that delays (i.e., snoozes) the remediation to a later time. The client device 50 may be operable to set a timer or other mechanism for reminding the user to perform the recommended remediation action(s).

In a second portion 580 of the window 506 of graphical user interface 50, further recommendations and/or suggestions for preventing a recurrence of the same problem may be provided. These further recommendations and/or suggestions for the remediation and prevention of further occurrences may be maintained in a database that is specific to the particular user and/or the particular swimming pool. The database (not shown in this example) may include a history of the user's monitoring results, past remediation, success of the past remediation, and recommendations/suggestions based on the history of the user's monitoring results. Additionally, a database may be maintained of other users' monitoring results, past remediation, success of the past remediation, and recommendations/suggestions based on the respective history of the user's monitoring results. The data from the other users may be used to determine the efficacy of remediation recommendations, remediation products, and the like.

A user can return to the previous window 502 of the graphical user interface 504 by selecting the back arrow control element 507.

In some variations, the water monitoring system 100 may enable the user to use, for example, a slider that is configured to adjust the testing and reporting frequencies to save or prolong the battery life of monitoring unit 40.

Advantageously, in some implementations, to supplement products, chemicals, and dosage amounts that have been used successfully in the past to address particular issues with the tested water, the present disclosed subject matter may include a crowd-sourced database of chemicals and products that discrete users have successfully used in connection with their pool, spa, hot tub, and the like. For example, when a user opts to use a product or brand name that is unknown or unrecognized but that includes a known chemical, active ingredient, and/or remediation agent, then the processing device will be capable of, initially, recommending a proper dosage (e.g., based on the chemical type in the unknown product, the concentration of the chemical, active ingredient, and/or remediation agent, and so forth) and, furthermore, storing that information in the crowd-sourced database of chemicals and products.

Referring back to the discussion of the second portion 580 of the graphical user interface, in some implementations, the processing device may be further configured and operable in gain more information about the product or brand from the user. Indeed, the processing device may be capable of querying the user as to, for example: the brand name of the product used, the active ingredient in the product used, the percentage or concentration of the active ingredient in the product used, the amount of the product added to water (i.e., the dosage amount), the measured change in the water property for the dosage amount, and so forth. These data may be saved to the crowd-sourced database of chemicals and products. Advantageously, when the crowd-sourced database of chemicals and products compiles a pre-determined number of user entries that provide identical or substantially identical entries of one or more of, for example: the brand name of the product, the concentration of the active ingredient(s) in the product, the dosage of the product used, and/or the results attained with the use, then the crowd-sourced database of chemicals and products may be able to add one or more of the following to the database: the brand name of the product, the active ingredient(s) in the product, the concentration of the active ingredient(s) in the product, the dosage used, the results attained data and so forth. The processing device may then use such data about the previously unknown or unrecognized product when making recommendations to other users with a similar problem.

Having described a system 100 and monitoring unit 40 for monitoring water conditions at a swimming pool, a method of monitoring the water conditions at the swimming pool and the interaction between the various elements of the water monitoring system 100 and device will now be described.

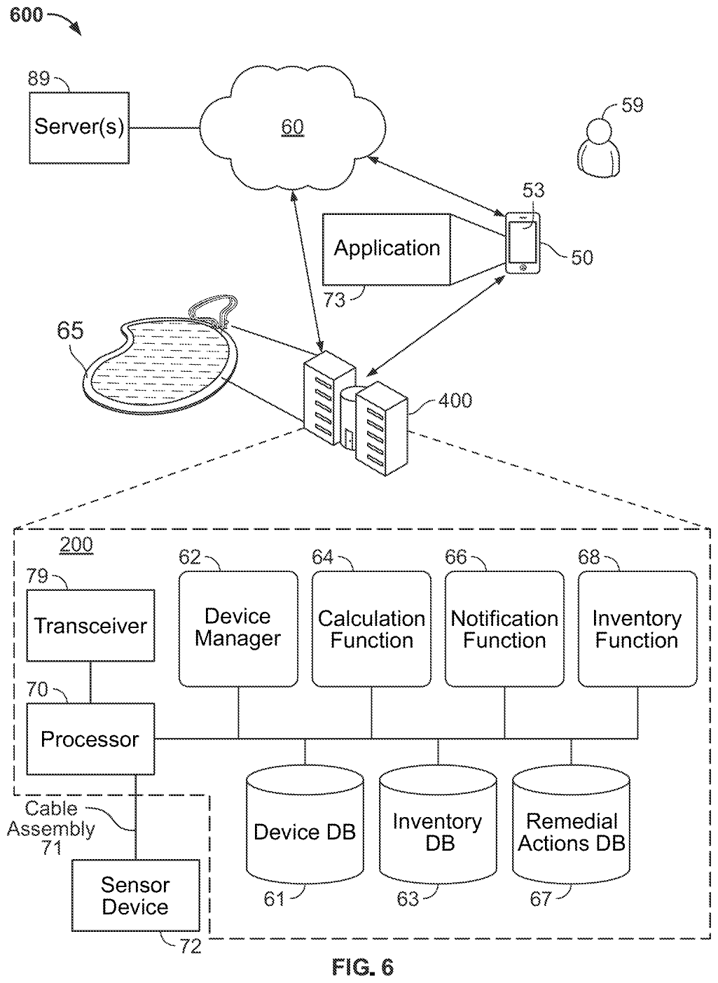

FIG. 6 shows an illustrative embodiment of a swimming pool monitoring system. In FIG. 6, the swimming pool monitoring system 600 includes a monitoring unit 40, a client device 50, a network 60, and swimming 65. The swimming pool monitoring system 600 provides functions for managing the chemistry of the water.

a monitoring unit 400 may include features like those described with reference to monitoring device 40 of FIG. 1. As such, a detailed description of similar features and details of monitoring device 400 may be omitted. The monitoring device 400 may include a surface based unit, such as 200, a submersible cable connection assembly 171, and a submerged sensing device 72. Similar to submerged sensing device 10 of FIG. 1, the submerged sensing device 72 is configured to measure water parameters, such as water temperature, pH, ORP and other parameters. In this example, the surface based unit 200 may be coupled to a first end of the submersible cable connection assembly 71, and the submerged sensing device 72 is coupled to a water-tight, second end of the submersible cable connection assembly 71. The surface base unit 200 may configured to receive at processor 70, the measured water parameters via the submersible cable connection assembly 71.

The client device 50 may be a smartphone, a dedicated control device, or smart remote control, or the like. For example, the client device 50 may include a computer application 73 stored in a memory (not shown) of the client device 50 and executable by a processor (not shown) of the client device 50.

The surface base unit 200 of the monitoring unit 400 may be operable to wirelessly communicate with the computer application 73 and the client device 50 includes a touchscreen 53 communicatively coupled to the computer application 73 via the client device processor (not shown). In an operational example of when the computer application is executing on the client device, the client device 50 may be operable to: receive the measured water parameters transmitted by the surface base unit 200; evaluate the measured water parameters; and present indications on the touchscreen 53 of the measured water parameters and evaluation results of the measured water parameters (See FIGS. 5A-C). In the example, the computer application 73 may include algorithms that evaluate the received measured water parameters with respect to industry standards for the respective water parameters, such as pH, ORP, water temperature, alkalinity, and the like, as well as historical values of the respective water parameters of the pool 65.

In a further operational example with regard to the evaluation of the water parameters, the pool water monitoring system 600 may utilize the communication network 60. The communication network 60 may be configured to communicate with a server 66 and the client device via the computer application 73 and/or the surface base unit 200 via processor 70 and transceiver 79 may be operable to communicate with the server 66 via the communication network 60. The server 66 may include additional data from a substantial number of users, chemical manufacturers, and research organizations as well as artificial intelligence algorithms to provide remedial action recommendations.

In some examples, the surface base unit 200 is configured to transfer measured water parameters to the external server 66 via the communication network 60. Alternatively, or in addition, the client device is configured to transfer measured water parameters and evaluation results of the measured water parameters to the external server 66 via the communication network 60. The computer application 73 may be operable to, in response to the transferred measured water parameters and/or evaluation results, receive remedial action messages (e.g., recommendations in varying degree of urgency, such as do now, do within one week, do this month, or the like) from the external server 66. The remedial action messages are based on the measured water parameters. The computer application 73 may cause the processor of the client device 50 to present the received remedial action messages on the touchscreen 53 of the client device 50.

The monitoring unit 40 may include the hardware previous described as well as software components maintained in memory and databases that may be deployed in one or more databases that may disposed locally or remotely, e.g., in one or more geographical locations. In some implementations, the monitoring unit 40 may include software components that include, for example, a device manager 62, a calculation function 64, a notification function 66, and an inventory function 68. The respective software components, 62, 64, 66 and 68 may be executed by a processor 70.

Furthermore, the monitoring unit 40 may include a number of databases that may include, for example, a device data database 61, an inventory data database 62, and a remedial action database 67. These databases 61, 63, 67 may reside in one or more physical data storage systems, such as a memory in the monitoring unit, memory in the client device 50, or connected to the network 60.

Advantageously, the monitoring unit 40 may be (e.g., wirelessly) connected to a respective client device 50 (e.g., via a respective Bluetooth connection) of a user 59. As previously described, the client device 50 may be structured and arranged to communicate with the monitoring unit 40 through one or more data communication networks 60 such as, for example, the Internet, cellular network, LAN, WAN, peer-to-peer networks (e.g., ad hoc peer-to-peer networks), or the like. A client application running on the client device 50 can be configured and operable to display one or more selective chemical parameters of a swimming pool 65 in which the submersible sensing device 10 is submerged and, furthermore, to display notifications or alerts (e.g., supply level or other user actions).

More particularly, a client device 50 may be structured and arranged to relay measurement data from a monitoring unit 40, for example, to the device manager 62. Advantageously, the device manager 62 may be configured and operable to store measurement data in the device data database 61. Using the measurement data, the monitoring unit 40 may be configured to determine parameters and to prepare notifications or alerts to be displayed on the client device 50 of a user 59 and, moreover, to transmit the data and notifications to the client application running on the client device 50.

The client application may then display the parameters and notifications in or on the display device of its user interface. In some instances, the client device 50 may annotate measurement data with images of the pool 65 or other indicators of the state of the pool water. These images may be synchronized with measurement data based, for example, on timestamps electronically associated with the images and the measurement data to increase accuracy of the measurements.

The calculation function 64 is a software component that, based on measurement data from a monitoring unit 40 submerged in the swimming pool 65, is configured to calculate an amount of a chemical that must be added to the swimming pool 65 to remediate any sensed problems with the chemistry or other problems associated with the water. For example, the calculation function 64 may be configured to estimate an amount (e.g., volume) of water in the swimming pool 65. Based on an expected impact (e.g., change in a measured parameter) of an amount of a discrete chemical added to the given volume of water, and based on data measured by the monitoring unit 40 after the chemical has been added to the pool 65, the calculation function 64 may be configured to measure or predict the actual impact on the water in the pool 65.

For example, if 16 ounces of acid are expected to lower the pH from 7.6 to 7.4 in 10,000 gallons of water, but the pH only drops from 7.6 to 7.5, then the calculation function 64 can estimate that the pool size is around 20,000 gallons. In this way, the calculation function 64 can continue this volume calculation every time chemicals are added to the pool 65 and refine its estimate of the pool's volume. For instance, if the instruction for the pool 65 is to add a desired dose of a chemical or active ingredient into the pool 65, but the calculation function 64 does not see the expected change in chemical readings, the calculation function 64 may be structured and arranged to adjust the estimate of the pool size; so that, the next time that a certain dosage is required, the instructions presented on the client device 50 can include an increased dosage. Advantageously, the calculation function 64 includes an AI learning function that can examine the impact of the dosage and, as a result, continue to improve its accuracy over time. In an example, the AI learning function is configured to track the water's response to chemical additions. Based on the output of the AI learning function, the interplay of chemicals and the water monitoring results from the added chemicals and the water's response can be used to learn ancillary information about the water. For example, if 1 ounce of chlorine is added to the water, the ORP is expected to rise by an estimated X millivolts (mV). However, if the ORP does not rise by the estimated mV, the processor executing the AI learning function may be configured to conclude that other factors in the water such as cyanuric acid are preventing the ORP rise, and may be configured to make a recommendation to adjust the cyanuric acid.

In addition, the AI learning function may be further utilized to receive additional data not only from the monitoring unit 40, but from water reports from the users as well as pool supply stores or other swimming pools utilizing another monitoring unit in the vicinity (e.g., city, county, area code, postal code, or the like) of the swimming pool monitored by monitoring unit 40. Using this additional data, the AI learning function may be configured to further refine the systems understanding of the swimming pool water.

When the calculation function 64 calculates an amount of a chemical needed for a swimming pool 65 based on measurement data of the pool by a monitoring unit 40, the calculation function 64 may be configured and operable to determine a user action corresponding to the calculated amount of the chemical. For instance, a user remedial action may require adding 8 ounces of pH Down to the pool 65. Accordingly, the device manager 62 may be configured to transmit a notification including the user remedial action to the client device 50. After receiving the notification, the client application can be configured and operable to present the user remedial action on the display device of the client device's user interface. Alternatively, instead of transmitting notifications of user remedial actions whenever a new user remedial action is determined by the calculation function 64, the monitoring unit 40 may be structured and arranged to combine several user remedial actions in a single notification and to transmit the combined action notification to the client device 50 of the user 59.

The calculation function 64 may be further structured and arranged to store each historic determined user action in the remedial actions database 67. The notification function 66 can collect user remedial actions (e.g., those stored in the remedial actions database 67) for a particular user 59 at a later time and send a notification including the combined user actions to the client device 50 of the user 59.

An additional feature of the presently disclosed subject matter is the ability to calibrate the sensors and/or monitors, for example, using the network 60. For example, the network 60 may be configured as a cloud platform having servers 66 and memory or data storage devices. For example, computer applications, such as computer application 73 implemented on the client device 50 may be operable, if data suggest that sensor pH readings are off by, say, (−) 0.2, to automatically compensate for the variation (i.e., the (−) 0.2) and adjust the data in the databases including any calculations that utilize the pH readings. To determine that any of the sensor or monitor readings are off, requiring calibration within the cloud, the system can compare the readings with those from another device, person, and/or store selling the system. For example, external test results (e.g., testing not based on the output of the monitoring unit 40) from a local lab, pool supply store, or the like of the swimming pools water chemistry may be used to calibrate sensor 20. Alternatively, the readings of a specific calibration test can be used to compare the data in the cloud with the results of the calibration test. For example, if a user brings a water sample into a local store and the store personnel determine a pH of 7.4 for the water sample but the sensor/monitor reads 7.2, the sensor/monitor can be calibrated to compensate for the 0.2 difference. Using a report provided by a pool supply store or another pool water testing facility, a user may use a printed report or digital report (containing water related values such as pH, ORP, cyanuric acid level, temperature, alkalinity, clarity, and the like) and enter that information from the report into a computer application (described in a later example) executing on a client device 50. Using the entered information, the monitoring unit 40 may be calibrated based on one or more of the entered values that were contained in the report. In addition, or alternatively, the store may also be able to enter the water related values into an authenticated web application via a server, such as server 66, that allows calibration values to be downloaded via wireless communication either via the client device 50 or a home network connection to the monitoring device 40.

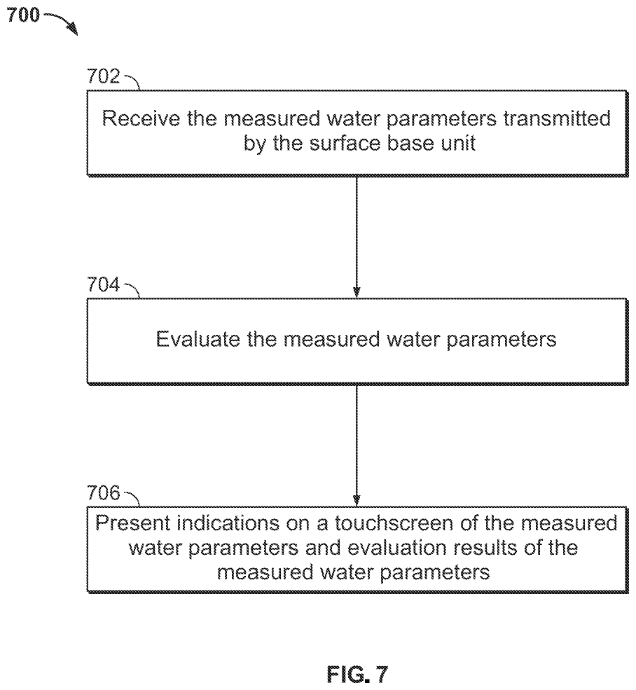

FIG. 7 illustrates a flowchart of an exemplary algorithm executed by the embodiments of a monitoring system as described herein.

As described herein a water monitoring system may include computer applications, such as computer application 73 executing on a client device 50. However, it is also envisioned that algorithms executed by the computer application 73 may also be executed by the processor 70 of the surface based unit 200, a processor of surface based unit 20, or by one or more of servers 66 as described above with reference to their respective examples. The water monitoring algorithm 700 may be implemented by a single processor or may be distributed between one or more of the processors, servers or the like as described here.

In step 702, a processor may be operable to receive measured water parameters transmitted by the surface base unit. As described herein, a sensing device may be operable to measure water parameters.

The measured water parameters may be evaluated by a processor or a server (704). The surface base unit may include a processor that is operable to evaluate the measured water parameters. For example, a processor of the surface base unit may compare to measured water parameters to reference parameters, such as a reference pH value known to provide comfortable and bacteria free swimming conditions, reference clarity standards, reference alkaline setting, and the like. Of course, the processor may be operable to perform calculations and the like using the measured water parameters when evaluating the measured water parameters. Alternatively, the surface base unit may be operable to transmit the measured water parameters to a client device and/or a server. For example, the surface base unit may communicate directly with the client device through Bluetooth or, if the client device is unavailable via Bluetooth, the surface base unit may utilize a communication network (via Wi-Fi or a cellular connection) to transmit the measured water parameters. The evaluation at 704 is to determine the quality of the water in the swimming pool.

After the evaluation of the measured water parameters, a processor, for example the processor of the client device while executing the computer application 73 may, at 706, present on a touchscreen (such as 507 in FIGS. 5A-C) indications of the measured water parameters and evaluation results of the measured water parameters.

Other steps, such as remedial actions, may be performed based on the received indications. Remedial actions may include adding water treatment chemicals to the water and repeating the process 700.

A computer application (also known as a program, software, software application, driver program, script, code, and so forth) may be written in any form of programming language, including compiled or interpreted languages, declarative or procedural languages. Moreover, the computer program may be deployed in any form, including as a stand-alone program or as a module, component, subroutine, object, or other unit suitable for use in a computing environment. A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus can also be implemented as, special purpose logic circuitry, e.g., a field programmable gate array (FPGA) or an application-specific integrated circuit (ASIC).

Processors suitable for the execution of a computer program include, by way of example, both general and special purpose microprocessors, and any one or more processors of any kind of digital computer. Generally, a processor will receive instructions and data from a read-only memory (ROM) and/or a random access memory (RAM). The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer may be embedded in another device, e.g., a smart phone, a smart watch, a mobile audio or video player, a game console, a tablet, a Global Positioning System (GPS) receiver, a portable storage device (e.g., a universal serial bus (USB) flash drive), and so forth.

Devices suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Implementations of the subject matter described in this specification may be implemented in a computing system that includes a back-end component, e.g., as a data server, or that includes a middleware component, e.g., an application server, or that includes a front-end component, e.g., a client computer having a graphical user interface or a web browser through which a user may interact with an implementation of the subject matter described in this specification, or any combination of one or more such back-end, middleware, or front-end components. The components of the system may be interconnected by any form or medium of digital data communication, e.g., a communication network.

A system of one or more computers may be configured to perform particular operations or actions by virtue of having software, firmware, hardware, or a combination of them installed on the system that in operation causes or cause the system to perform the actions. One or more computer programs may be configured to perform particular operations or actions by virtue of including instructions that, when executed by data processing apparatus, cause the apparatus to perform the actions.

While this specification contains many specific implementation details, these should not be construed as limitations on the scope of any disclosed subject matters or of what may be claimed, but rather as descriptions of features specific to particular implementations of particular disclosed subject matters. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations. Furthermore, it should be understood that the described program components and systems may, optionally, be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A monitoring unit for monitoring the condition of pool water, the monitoring device comprising:
a sensing device configured to be submerged in the pool water, the sensing device including one or more water parameter sensors, wherein the one or more water parameter sensors are operable to measure at least one parameter of the water;
a surface base unit disposed to be out of the pool water, the surface base unit including electronic components configured to receive a measurement data of at least one parameter of the pool water from the sensing device and to wirelessly transmit the measurement; and
a submersible cable connection assembly removably coupled to the surface base unit outside of the pool water and permanently coupled to the sensing device that is submerged in the pool water.

2. The monitoring unit of claim 1, wherein the submersible cable connection assembly includes:
a first end that is electrically coupled via a waterproof connection to the sensing device; and
a second end that terminates in a watertight electrical connector operable to be removably coupled to the surface base unit.

3. The monitoring unit of claim 1, wherein the surface base unit includes:
an upper housing portion;
an inner annular portion;
an inner top portion; and
a lower housing portion, wherein the upper housing, the inner annular portion, the inner top portion, and the lower housing are structured to provide a water-tight seal when operatively coupled together.

4. The monitoring unit of claim 3, wherein the inner top portion of the surface base unit includes:
a power source housing area configured to accommodate a power source and a power source connection bus, wherein the power source connection bus includes a power supply connector to supply power to other components of the surface base unit.

5. The monitoring unit of claim 3, wherein the lower housing portion of the surface base unit is configured to house the surface base unit circuitry.

6. The monitoring unit of claim 3, wherein the lower housing portion of the surface base unit includes:
an inner data port connection coupled to surface base circuitry within the lower housing portion; and
an outer data port connector, wherein the outer data port connector is configured to electrically couple the surface base unit to the submersible cable connection assembly.

7. The monitoring unit of claim 6, wherein the outer data port connector comprises:
a cover configured to provide a watertight seal when the submersible cable connection assembly is removed from the surface base unit.

8. The monitoring unit of claim 3, wherein the lower housing portion of the surface base unit includes:
an attachment area configured to receive attachment devices enabling the surface base unit to be affixed to structures of a body holding the pool water.

9. The monitoring unit of claim 3, wherein the inner annular portion is configured as a circular arc having an opening operable to allow space for an inner data port connection to be accessible to an outer data port connector.

10. The monitoring unit of claim 3, wherein the inner annular portion is further configured with fixation openings to enable fastening devices to be inserted through the fixation openings and into securing points in the lower housing portion.

11. The monitoring unit of claim 10, wherein when the fastening devices are inserted through the fixation openings and into the securing points, the inner annular portion is affixed to the lower housing portion.

12. The monitoring unit of claim 1, wherein the upper housing portion is configured to removably couple to the inner annular portion of the surface base unit to conceal fixation openings formed in the inner annular portion.

13. A pool water monitoring system, comprising:
a monitoring unit comprising a surface based unit, a submersible cable connection assembly, and a submerged sensing device, wherein:
the submerged sensing device is configured to measure water parameters,
the surface based unit is coupled to a first end of the submersible cable connection assembly, and the submerged sensing device is coupled to a water-tight, second end of the submersible cable connection assembly, and is configured to receive the measured water parameters via the submersible cable connection; and
a computer application executable on a client device, wherein the surface base unit of the monitoring unit is operable to wirelessly communicate with the computer application and the client device includes a touchscreen communicatively coupled to the computer application, and when the computer application is executing on the client device, wherein the client device is operable to:
receive the measured water parameters transmitted by the surface base unit;
evaluate the measured water parameters; and
present indications on the touchscreen of the measured water parameters and evaluation results of the measured water parameters.

14. The pool water monitoring system of claim 13, wherein the submersible cable connection assembly includes:
a first end that is electrically coupled via a waterproof connection to the sensing device; and
a second end that terminates in a watertight electrical connector operable to be removably coupled to the surface base unit.

15. The pool water monitoring system of claim 13, wherein the surface base unit includes:
an upper housing portion;
an inner annular portion;
an inner top portion; and
a lower housing portion, wherein the upper housing, the inner annular portion, the inner top portion, and the lower housing are structured to provide a substantially water-tight seal when operatively coupled together.

16. The pool water monitoring system of claim 15, wherein the lower housing portion of the surface base unit is configured to house the surface base unit circuitry.

17. The pool water monitoring system of claim 15, wherein the lower housing portion of the surface base unit includes:
an inner data port connection coupled to surface base circuitry within the lower housing portion; and
an outer data port connector, wherein the outer data port connector is configured to electrically couple the surface base unit to the submersible cable connection assembly.

18. The pool water monitoring system of claim 13, further comprising:
a communication network configured to communicate with a server, wherein the client device and/or the surface unit is operable to communicate with the server via the communication network.

19. The pool water monitoring system of claim 18, wherein the surface unit is configured to transfer measured water parameters to the server.

20. The pool water monitoring system of claim 18, wherein the client device is configured to:
transfer measured water parameters and evaluation results of the measured water parameters to the server;
in response to the transferred measured water parameters and/or evaluation results, receive remedial action messages from the server, wherein the remedial action messages are based on the measured water parameters; and
present the received remedial action messages on the touchscreen of the client device.
